(19) United States
(12) Patent Application Publication
(10) Pub. No.: US 2014/0193484 A1
(43) Pub. Date: Jul. 10, 2014

(54) INFLUENZA VIRUS IMMUNOGENIC COMPOSITIONS AND USES THEREOF

(71) Applicants: Sylvie Carine Bertholet Girardin, Siena (IT); Andrew Geall, Littleton, MA (US)

(72) Inventors: Sylvie Carine Bertholet Girardin, Siena (IT); Andrew Geall, Littleton, MA (US)

(21) Appl. No.: 14/152,397

(22) Filed: Jan. 10, 2014

Related U.S. Application Data
(60) Provisional application No. 61/751,077, filed on Jan. 10, 2013.

(51) Int. Cl. A61K 39/145; A61K 9/127

(52) U.S. Cl. CPC A61K 39/145; A61K 9/127
USPC 424/450; 424/210.1

(57) ABSTRACT

Immunogenic compositions comprise a RNA component and a polypeptide component. The RNA component is a self-replicating RNA. The polypeptide component comprises an epitope from an influenza virus antigen (the first epitope), and the RNA component encodes a polypeptide which also comprises an epitope from an influenza virus antigen (the second epitope). Delivery of epitopes in these two different manners can enhance the immune response to influenza virus as compared to immunization with the RNA or the polypeptide alone.

### RELATED APPLICATION

This patent application claims the benefit of U.S. Provisional Application No. 61/751,077, filed Jan. 10, 2013, the complete contents of which are incorporated herein by reference.

### STATEMENT OF GOVERNMENT SUPPORT

This invention was made in part with Government support under Agreement No. HR0011-12-3-0001 awarded by The Defense Advanced Research Projects Agency (DARPA). The Government has certain rights in the invention.

### TECHNICAL FIELD

This invention is in the field of non-viral delivery of mixtures of RNA and proteins for immunisation against influenza virus.

### BACKGROUND OF THE INVENTION

Nucleic acid based vaccines are an attractive approach to immunisation. For instance, WO2012/006369 discloses the use of self-replicating RNA molecules for this purpose, and WO2013/006842 describes an approach in which a first polypeptide is co-delivered with a self-replicating RNA which encodes a second polypeptide. The two polypeptides are from the same pathogen, but they do not need to be the same polypeptide. Thus WO2013/006842 discloses that they can share an epitope or can have different epitopes, but they must be from the same pathogen. This provides a composition that delivers epitopes in two different forms—a first epitope from a pathogen, in RNA-coded form; and a second epitope from the same pathogen, in polypeptide form—which can enhance the immune response to the pathogen, as compared to immunization with RNA alone, or polypeptide alone.

It is an object of the invention to provide further approaches to immunisation which utilise self-replicating RNA.

### SUMMARY OF THE INVENTION

This invention generally relates to immunogenic compositions that comprise a self-replicating RNA component and a polypeptide component. The polypeptide component comprises an epitope from an influenza virus antigen (the first epitope), and the self-replicating RNA component encodes a polypeptide which also comprises an epitope from an influenza virus antigen (the second epitope). Immunogenic compositions that deliver epitopes in these two different manners can enhance the immune response to an influenza virus, as compared to immunization with the RNA alone, or the polypeptide alone.

Thus the invention provides an immunogenic composition comprising (a) a polypeptide that comprises an epitope from an influenza virus antigen, and (b) a self-replicating RNA which encodes a polypeptide that comprises an epitope from an influenza virus antigen.

The invention also provides a kit comprising (a) a first kit component comprising a polypeptide that comprises an epitope from an influenza virus antigen, and (b) a second kit component comprising a self-replicating RNA which encodes a polypeptide that comprises an epitope from an influenza virus antigen.

The invention also provides methods for treating and/or preventing influenza virus disease and/or infection, methods for inducing an immune response against influenza virus, and methods for vaccinating a subject, by co-delivery of a self-replicating RNA molecule and a polypeptide molecule as described above (co-administration).

The invention also provides methods for treating and/or preventing influenza virus disease and/or infection, methods for inducing an immune response against influenza virus, and methods for vaccinating a subject, by sequential administration of a self-replicating RNA molecule and a polypeptide molecule as described above (prime-boost).

In a first embodiment of the invention, the first and second epitopes are both from influenza hemagglutinin.

In a second embodiment, the first and second epitopes are both from influenza A virus. Ideally, they are both from influenza A virus strains having the same HA subtype e.g. both from an influenza A virus of H5 subtype. In certain aspects of this embodiment, the first and second epitopes are both hemagglutinin epitopes from the same HA subtype.

In a third embodiment, the first epitope and the second epitope are both from influenza B virus. In certain aspects of this embodiment, the first and second epitopes are both hemagglutinin epitopes from influenza B virus.

In a fourth embodiment, the first epitope and the second epitope are both from influenza B virus strains in the B/Yamagata/16/88-like lineage. In certain aspects of this embodiment, the first and second epitopes are both hemagglutinin epitopes from an influenza B virus strain in the B/Yamagata/16/88-like lineage.

In a fifth embodiment, the first epitope and the second epitope are both from influenza B virus strains in the B/Victoria/2/87-like lineage. In certain aspects of this embodiment, the first and second epitopes are both hemagglutinin epitopes from an influenza B virus strain in the B/Victoria/2/87-like lineage.

### BRIEF DESCRIPTION OF DRAWINGS

FIG. 1 shows HI titers (GMT) in experimental groups at day 72. The “ns” bars show groups whose titers were not significantly different, whereas ** indicates a significant difference.

FIG. 2 shows the frequency of pentamer-positive cells (% of CD8+ CD44h T cells).

FIG. 3 shows CD8 T cells (% of antigen-specific CD8+ T cells, IFNγ).

### DETAILED DESCRIPTION OF THE INVENTION

This invention generally relates to immunogenic compositions that comprise a RNA component and a polypeptide component. The RNA component is a self-replicating RNA, as described in more detail below. The polypeptide component comprises an epitope from an influenza virus antigen (the first epitope), and the RNA component encodes a polypeptide which also comprises an epitope from an influenza virus antigen (the second epitope). As noted in WO2013/006842, immunogenic compositions that deliver epitopes in these two different manners can enhance the immune response to a pathogen (influenza virus), as compared to immunization with the RNA alone, or the polypeptide alone.

Thus the invention provides an immunogenic composition comprising (a) a polypeptide that comprises an epitope from an influenza virus antigen, and (b) a self-replicating RNA which encodes a polypeptide that comprises an epitope from an influenza virus antigen.

The invention also provides a kit comprising (a) a first kit component comprising a polypeptide that comprises an epitope from an influenza virus antigen, and (b) a second kit component comprising a self-replicating RNA which encodes a polypeptide that comprises an epitope from an influenza virus antigen.

The invention also provides methods for treating and/or preventing influenza virus disease and/or infection, methods for inducing an immune response against influenza virus, and methods for vaccinating a subject, by co-delivery of a RNA molecule and a polypeptide molecule as described above (co-administration).

The invention also provides methods for treating and/or preventing influenza virus disease and/or infection, methods for inducing an immune response against influenza virus, and methods for vaccinating a subject, by sequential administration of a RNA molecule and a polypeptide molecule as described above (prime-boost).

In a first embodiment of the invention, the first and second epitopes are both from influenza hemagglutinin.

In a second embodiment, the first and second epitopes are both from influenza A virus. Ideally, they are both from influenza A virus strains having the same HA subtype e.g. both from an influenza A virus of H5 subtype. In certain aspects, the first and second epitopes are both hemagglutinin epitopes from the same HA subtype.

In a third embodiment, the first epitope and the second epitope are both from influenza B virus. In certain aspects, the first and second epitopes are both hemagglutinin epitopes from influenza B virus.

In a fourth embodiment, the first epitope and the second epitope are both from influenza B virus strains in the B/Yamagata/16/88-like lineage. In certain aspects, the first and second epitopes are both hemagglutinin epitopes from an influenza B virus strain in the B/Yamagata/16/88-like lineage.

In a fifth embodiment, the first epitope and the second epitope are both from influenza B virus strains in the B/Victoria/2/87-like lineage. In certain aspects, the first and second epitopes are both hemagglutinin epitopes from an influenza B virus strain in the B/Victoria/2/87-like lineage.

### Influenza Virus Antigens

Influenza virus has three types—A, B, and C. Influenza A virus is the most common flu virus infecting humans, animals, and birds. Influenza B virus infection mostly occurred in humans. Infection of influenza C virus does not cause any severe symptom in human or mammals.

Influenza virus strains can change from season to season. In the current inter-pandemic period, current seasonal trivalent vaccines include two influenza A strains (one H1N1 strain and one H3N2 strain) and one influenza B strain. Characteristics of a pandemic influenza strain are: (a) it contains a new hemagglutinin compared to the hemagglutinins in currently-circulating human strains, i.e. one that has not been evident in the human population for over a decade (e.g. H2), or has not previously been seen at all in the human population (e.g. H5, H6, H9, that have generally been found only in bird populations), such that the vaccine recipient and the general human population are immunologically naïve to the strain's hemagglutinin; (b) it is capable of being transmitted horizontally in the human population; and (c) it is pathogenic to humans. Pandemic strains are commonly H2, H5, H7 or H9 subtype influenza A virus strains e.g. H5N1, H5N3, H9N2, H2N2, H7N1 and H7N7 strains. Within the H5 subtype, a virus may fall into different clades.

Influenza A virus currently displays seventeen HA (hemagglutinin) subtypes: H1, H2, H3, H4, H5, H6, H7, H8, H9, H10, H11, H12, H13, H14, H15, H16, and H17. It also displays nine NA (neuraminidase) subtypes: N1, N2, N3, N4, N5, N6, N7, N8, and N9.

Influenza B virus currently does not display different HA subtypes, but influenza B virus strains do fall into two distinct lineages. These lineages emerged in the late 1980s and have HAs which can be antigenically and/or genetically distinguished from each other [Rota et al. (1992) J Gen Virol 73:2737-42]. Current influenza B virus strains are either B/Victoria/2/87-like or B/Yamagata/16/88-like. Strains in the two lineages are usually distinguished antigenically, but differences in amino acid sequences have also been described for distinguishing them e.g. B/Yamagata/16/88-like strains often (but not always) have HA proteins with deletions at amino acid residue 164, numbered relative to the ‘Lee40’ HA sequence (GenBank sequence GI:325176).

In some embodiments, the first and second epitopes are both from influenza virus hemagglutinins. For example: (a) the first epitope could be from an influenza A virus hemagglutinin and the second epitope could be from an influenza B virus hemagglutinin; (b) the first epitope could be from an influenza A virus hemagglutinin and the second epitope could be from an influenza A virus hemagglutinin; or (c) the first epitope could be from an influenza B virus hemagglutinin and the second epitope could be from an influenza B virus hemagglutinin. Ideally, the two epitopes are both from the same influenza virus type e.g. both from A, or both from B.

In embodiments where the first and second epitopes are both from influenza A virus, ideally they are both hemagglutinin epitopes, and are from influenza A virus strains having the same HA subtype. For example, both epitopes could be from a H1 hemagglutinin, a H2 hemagglutinin, a H3 hemagglutinin, a H4 hemagglutinin, a H5 hemagglutinin, a H6 hemagglutinin, a H7 hemagglutinin, a H8 hemagglutinin, a H9 hemagglutinin, a H10 hemagglutinin, a H11 hemagglutinin, a H12 hemagglutinin, a H13 hemagglutinin, a H14 hemagglutinin, a H15 hemagglutinin, a H16 hemagglutinin, or a H17 hemagglutinin. In certain aspects, both epitopes are from a H1 hemagglutinin, a H3 hemagglutinin, or a H5 hemagglutinin.

In embodiments where the first and second epitopes are both from influenza B virus, ideally they are both hemagglutinin epitopes, and are from influenza B virus strains in the same lineage. For example, both epitopes could be from a strain in the B/Victoria/2/87-like lineage, or both epitopes could be from a strain in the B/Yamagata/16/88-like lineage.

In all embodiments, usually the first epitope and the second epitope are from the same influenza virus strain. In certain aspects, the first epitope and the second epitope are the same epitope.

### The Self-Replicating RNA

An immunogenic composition of the invention includes a RNA component which encodes a polypeptide which comprises an epitope from an influenza virus antigen (the second epitope). After administration to a subject, the RNA is translated inside a cell to provide an influenza virus polypeptide in situ.

The RNA should be +-stranded, and so it can be translated by cells without needing any intervening replication steps such as reverse transcription. Advantageously, it can also bind to TLR7 receptors expressed by immune cells, thereby initiating an adjuvant effect. Preferred +-stranded RNAs are self-replicating. A self-replicating RNA molecule (replicon) can, when delivered to a vertebrate cell even without any proteins, lead to the production of multiple daughter RNAs by transcription from itself (via an antisense copy which it generates from itself). A self-replicating RNA molecule is thus typically a +-strand molecule which can be directly translated after delivery to a cell, and this translation provides a RNA-dependent RNA polymerase which then produces both antisense and sense transcripts from the delivered RNA. Thus the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the polypeptide. The overall results of this sequence of transcriptions is a huge amplification in the number of the introduced replicon RNAs and so the encoded polypeptide becomes a major polypeptide product of the cells.

One suitable system for achieving self-replication is to use an alphavirus-based RNA replicon. These +-stranded replicons are translated after delivery to a cell to give of a replicase (or replicase-transcriptase). The replicase is translated as a polyprotein which auto-cleaves to provide a replication complex which creates genomic −-strand copies of the +-strand delivered RNA. These −-strand transcripts can themselves be transcribed to give further copies of the +-stranded parent RNA and also to give a subgenomic transcript which encodes the polypeptide. Translation of the subgenomic transcript thus leads to in situ expression of the polypeptide by the infected cell. Suitable alphavirus replicons can use a replicase from a sindbis virus, a semliki forest virus, an eastern equine encephalitis virus, a Venezuelan equine encephalitis virus, etc. Mutant or wild-type viruses sequences can be used e.g. the attenuated TC83 mutant of VEEV has been used in replicons (WO2005/113782).

A preferred self-replicating RNA molecule thus encodes (i) a RNA-dependent RNA polymerase which can transcribe RNA from the self-replicating RNA molecule and (ii) the polypeptide of interest. The polymerase can be an alphavirus replicase e.g. comprising one or more of alphavirus proteins nsP1, nsP2, nsP3 and nsP4.

Whereas natural alphavirus genomes encode structural virion proteins in addition to the non-structural replicase polyprotein, it is preferred that a self-replicating RNA molecule of the invention does not encode alphavirus structural proteins. Thus a preferred self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the invention and their place is taken by gene(s) encoding the polypeptide of interest, such that the subgenomic transcript encodes the polypeptide rather than the structural alphavirus virion proteins.

Thus a self-replicating RNA molecule useful with the invention may have two open reading frames. The first (5′) open reading frame encodes a replicase; the second (3′) open reading frame encodes a polypeptide. In some embodiments the RNA may have additional (e.g. downstream) open reading frames e.g. to encode further polypeptides (see below) or to encode accessory polypeptides.

A self-replicating RNA molecule can have a 5′ sequence which is compatible with the encoded replicase.

The self-replicating RNA molecule may be derived from or based on a virus other than an alphavirus, in particular, a positive-stranded RNA virus, and particularly a picornavirus, flavivirus, rubivirus, pestivirus, hepacivirus, calicivirus, or coronavirus. Alphaviruses are preferred, though, and suitable wild-type alphavirus sequences are well-known and are available from sequence depositories, such as the American Type Culture Collection, Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375). Chimeric alphavirus replicons which include components from multiple different alphaviruses may also be useful.

Self-replicating RNA molecules can have various lengths but they are typically 5,000-25,000 nucleotides long e.g. 8,000-15,000 nucleotides, or 9,000-12,000 nucleotides. Thus the RNA is longer than seen in siRNA delivery.

A RNA molecule useful with the invention may have a 5′ cap (e.g. a 7-methylguanosine). This cap can enhance in vivo translation of the RNA.

The 5′ nucleotide of a RNA molecule useful with the invention may have a 5′ triphosphate group. In a capped RNA this may be linked to a 7-methylguanosine via a 5′-to-5′ bridge. A 5′ triphosphate can enhance RIG-I binding and thus promote adjuvant effects.

A RNA molecule may have a 3′ poly-A tail. It may also include a poly-A polymerase recognition sequence (e.g. AAUAAA) near its 3′ end.

A RNA molecule useful with the invention will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

A RNA molecule useful with the invention can conveniently be prepared by in vitro transcription (IVT). IVT can use a (cDNA) template created and propagated in plasmid form in bacteria, or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

As discussed in WO2011/005799, the self-replicating RNA can include (in addition to any 5′ cap structure) one or more nucleotides having a modified nucleobase. For instance, a self-replicating RNA can include one or more modified pyrimidine nucleobases, such as pseudouridine and/or 5-methylcytosine residues. In some embodiments, however, the RNA includes no modified nucleobases, and may include no modified nucleotides i.e. all of the nucleotides in the RNA are standard A, C, G and U ribonucleotides (except for any 5′ cap structure, which may include a 7′-methylguanosine). In other embodiments, the RNA may include a 5′ cap comprising a 7′-methylguanosine, and the first 1, 2 or 3 5′ ribonucleotides may be methylated at the 2′ position of the ribose.

A RNA used with the invention ideally includes only phosphodiester linkages between nucleosides, but in some embodiments it can contain phosphoramidate, phosphorothioate, and/or methylphosphonate linkages.

The RNA encodes a polypeptide which comprises an epitope from an influenza virus antigen, as described in more detail above. The RNA ideally encodes a polypeptide comprising a fragment of an influenza virus hemagglutinin. It can encode a soluble cytosolic antigen, rather than a membrane-tethered or secreted antigen (although the cell may present the cytosolic antigen on the cell surface as part of immune processing). In situ expression of the polypeptide will elicit an anti-influenza immune response. For instance, it can lead to the production of antibodies which recognise an influenza virion e.g. antibodies which bind to virion-surface hemagglutinin. Ideally the elicited antibodies are neutralising or protective antibodies.

### The Polypeptide Component

An immunogenic composition of the invention includes a polypeptide component, and the polypeptide comprises an epitope from an influenza virus antigen (the first epitope).

The polypeptide component can be a single polypeptide, but can also be a multi-chain polypeptide structure (such as a polypeptide complex e.g., a complex formed by two or more proteins), a multimeric protein (e.g. trimeric hemagglutinin), or a large polypeptide structure, such as a VLP (virus-like particle).

Ideally, the polypeptide in the composition (the first polypeptide), and the polypeptide encoded by the self-replicating RNA (the second polypeptide), share at least one epitope. They can share many epitopes, particularly when the two polypeptides are long (e.g. longer than 80aa) and each include multiple epitopes.

In certain embodiments, the first polypeptide and the second polypeptide share at least 2, at least 3, at least 4, or at least 5 B-cell and/or T-cell epitopes. In certain embodiments, the first and second polypeptides share at least one immunodominant epitope. In certain embodiments, the first and second polypeptides share the same immunodominant epitope(s), or the same primary immunodominant epitope.

Usually, the first and second polypeptides share a common amino acid sequence e.g. the first and second polypeptides are identical, the first polypeptide is a fragment of the second polypeptide, the second polypeptide is a fragment of the first polypeptide, the first polypeptide is a fusion of a core sequence to a first fusion partner and the second polypeptide is a fusion of a cre sequence to a second fusion partner, etc. The common amino acid sequence ideally includes multiple epitopes, and it can be 80 amino acids or longer e.g. ≧100aa, ≧120aa, ≧140aa, ≧160aa, ≧180aa, ≧200aa, ≧220aa, ≧240aa, ≧260aa, ≧280aa, ≧300a a, ≧320aa, ≧340aa, ≧360aa, ≧380aa, ≧400aa, or more. The common amino acid sequence can comprise a complete HA1 hemagglutinin subunit, or an immunogenic fragment thereof.

In some embodiments, the first and second polypeptides have at least x % amino acid sequence identity to each other, where the value of x is 80, 85, 90, 92, 94, 95, 96, 97, 98, or 99. If one polypeptide is shorter than the other, the sequence identity should be calculated across the length of the shorter polypeptide. References to a percentage sequence identity between two amino acid sequences means that, when aligned, that percentage of amino acids are the same in comparing the two sequences. This alignment and the percent homology or sequence identity can be determined using software programs known in the art. A preferred alignment is determined by the Smith-Waterman homology search algorithm using an affine gap search with a gap open penalty of 12 and a gap extension penalty of 2, BLOSUM matrix of 62.

In addition to influenza-derived amino acid sequences, the polypeptide may include additional sequences, such as a sequence to facilitate expression, production, purification or detection (e.g., a poly-His sequence, a tag, etc.).

The polypeptide will usually be isolated or purified. Thus, it is not be associated with molecules with which they are normally, if applicable, found in nature.

Polypeptides will usually be prepared by expression in a recombinant host system. Suitable recombinant host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster), avian cells (e.g., chicken, duck, and geese), bacteria (e.g., E. coli, Bacillus subtilis, and Streptococcus spp.), yeast cells (e.g., Saccharomyces cerevisiae, Candida albicans, Candida maltosa, Hansenual polymorphs, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophile) or combinations thereof. Many suitable insect cells and mammalian cells are well-known in the art. Suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Suitable mammalian cells include, for example, Chinese hamster ovary (CHO) cells, human embryonic kidney cells (HEK293 cells, typically transformed by sheared adenovirus type 5 DNA), NIH-3T3 cells, 293-T cells, Vero cells, HeLa cells, PERC.6 cells (ECACC deposit number 96022940), Hep G2 cells, MRC-5 (ATCC CCL-171), WI-38 (ATCC CCL-75), fetal rhesus lung cells (ATCC CL-160), Madin-Darby bovine kidney (“MDBK”) cells, Madin-Darby canine kidney (“MDCK”) cells (e.g., MDCK (NBL2), ATCC CCL34; or MDCK 33016, DSM ACC 2219), baby hamster kidney (BHK) cells, such as BHK21-F, HKCC cells, and the like. Suitable avian cells include, for example, chicken embryonic stem cells (e.g., EBx® cells), chicken embryonic fibroblasts, chicken embryonic germ cells, duck cells (e.g., AGE1.CR and AGE1.CR.pIX cell lines which are described, for example, in Vaccine 27:4975-4982 (2009) and WO2005/042728), EB66 cells, and the like.

Suitable insect cell expression systems, such as baculovirus systems, are known to those of skill in the art and described in, e.g., Summers and Smith, Texas Agricultural Experiment Station Bulletin No. 1555 (1987). Materials and methods for baculovirus/insert cell expression systems are commercially available in kit form. Similarly, bacterial and mammalian cell expression systems are also known in the art.

Recombinant constructs encoding a polypeptide can be prepared in suitable vectors using conventional methods. A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable baculovirus expression vector, such as pFastBac (Invitrogen), is used to produce recombinant baculovirus particles. The baculovirus particles are amplified and used to infect insect cells to express recombinant protein. For expression in mammalian cells, a vector that will drive expression of the construct in the desired mammalian host cell (e.g., Chinese hamster ovary cells) is used.

Polypeptides can be purified using any suitable methods. For example, methods for purifying polypeptides by immunoaffinity chromatography are known in the art. Suitable methods for purifying desired proteins including precipitation and various types of chromatography, such as hydrophobic interaction, ion exchange, affinity, chelating and size exclusion are well-known in the art. Suitable purification schemes can be created using two or more of these or other suitable methods. If desired, the polypeptides can include a “tag” that facilitates purification, such as an epitope tag or a His-tag. Such tagged polypeptides can conveniently be purified, for example from conditioned media, by chelating chromatography or affinity chromatography.

### Delivery Systems

Although RNA can be delivered as naked RNA (e.g. merely as an aqueous solution of RNA), to enhance entry into cells and also subsequent intercellular effects, a RNA molecule is preferably administered in combination with a delivery system, such as a particulate or emulsion delivery system. Thus, in addition to the polypeptide and RNA components, compositions of the invention can include additional components, such as lipids, polymers or other compounds which can facilitate entry of RNA into target cells. Many delivery systems are well known to those of skill in the art.

The RNA may be introduced into cells by receptor-mediated endocytosis e.g., U.S. Pat. No. 6,090,619, Wu & Wu (1988) J. Biol. Chem., 263:14621, and Curiel et al. (1991) PNAS USA 88:8850. U.S. Pat. No. 6,083,741 discloses introducing an exogenous nucleic acid into mammalian cells by associating it with a polycation moiety (e.g., poly-L-lysine having 3-100 lysine residues), which is itself coupled to an integrin receptor-binding moiety (e.g., a cyclic peptide having a RGD sequence).

The RNA molecule can be delivered into cells via amphiphiles e.g. U.S. Pat. No. 6,071,890. Typically, a nucleic acid molecule may form a complex with the cationic amphiphile. Mammalian cells contacted with the complex can readily take it up.

Three particularly useful delivery systems are (i) liposomes (ii) non-toxic and biodegradable polymer microparticles (iii) cationic submicron oil-in-water emulsions.

### Liposomes

Various amphiphilic lipids can form bilayers in an aqueous environment to encapsulate a RNA-containing aqueous core as a liposome. These lipids can have an anionic, cationic or zwitterionic hydrophilic head group. Formation of liposomes from anionic phospholipids dates back to the 1960s, and cationic liposome-forming lipids have been studied since the 1990s. Some phospholipids are anionic whereas other are zwitterionic and others are cationic. Suitable classes of phospholipid include, but are not limited to, phosphatidylethanolamines, phosphatidylcholines, phosphatidylserines, and phosphatidyl-glycerols, and some useful phospholipids are listed in Table 1. Useful cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are DPPC, DOPC and dodecylphosphocholine. Other useful lipids are disclosed in WO2012/031046. The lipids can be saturated or unsaturated. The use of at least one unsaturated lipid for preparing liposomes is preferred. If an unsaturated lipid has two tails, both tails can be unsaturated, or it can have one saturated tail and one unsaturated tail.

In certain embodiments, liposomes comprise a lipid having a pKa in the range of 5.0 to 7.6 (e.g. 5.7 to 5.9), in particularly a lipid having a tertiary amine (see WO2012/006378).

Liposomes can be formed from a single lipid or from a mixture of lipids. A mixture may comprise (i) a mixture of anionic lipids (ii) a mixture of cationic lipids (iii) a mixture of zwitterionic lipids (iv) a mixture of anionic lipids and cationic lipids (v) a mixture of anionic lipids and zwitterionic lipids (vi) a mixture of zwitterionic lipids and cationic lipids or (vii) a mixture of anionic lipids, cationic lipids and zwitterionic lipids. Similarly, a mixture may comprise both saturated and unsaturated lipids. For example, a mixture may comprise DSPC (zwitterionic, saturated), Dlin DMA (cationic, unsaturated), and/or DMG (anionic, saturated). Where a mixture of lipids is used, not all of the component lipids in the mixture need to be amphiphilic e.g. one or more amphiphilic lipids can be mixed with cholesterol.

The hydrophilic portion of a lipid can be PEGylated (i.e. modified by covalent attachment of a polyethylene glycol). This modification can increase stability and prevent non-specific adsorption of the liposomes. For instance, lipids can be conjugated to PEG using techniques such as those disclosed in WO2005/121348 and in Heyes et al. (2005) J Controlled Release 107:276-87. Various lengths of PEG can be used e.g. between 0.5-8 kDa, between 1-3 kDa (WO2012/031043), between 3-11 kDa.

A mixture of DSPC, Dlin DMA, PEG-DMG and cholesterol is used in the examples. These can be made as disclosed in WO2012/006376.

Liposomes are usually divided into three groups: multilamellar vesicles (MLV); small unilamellar vesicles (SUV); and large unilamellar vesicles (LUV). MLVs have multiple bilayers in each vesicle, forming several separate aqueous compartments. SUVs and LUVs have a single bilayer encapsulating an aqueous core; SUVs typically have a diameter ≦50 nm, and LUVs have a diameter >50 nm. Liposomes useful with of the invention are ideally LUVs with a diameter in the range of 50-220 nm. For a composition comprising a population of LUVs with different diameters: (i) at least 80% by number should have diameters in the range of 20-220 nm, (ii) the average diameter (Zav, by intensity) of the population is ideally in the range of 40-200 nm, and/or (iii) the diameters should have a polydispersity index <0.2.

Liposomes with a diameter in the range of 60-180 nm can be particularly useful (WO2012/030901), such as those with a diameter in the range of 80-160 nm. For a composition comprising a population of liposomes with different diameters: (i) at least 80% by number of the liposomes should have diameters in the range of 60-180 nm, and in particular 80-160 nm, and/or (ii) the average diameter (by intensity e.g. Z-average) of the population is ideally in the range of 60-180 nm, and particularly 80-160 nm.

Apparatuses for determining the average particle diameter in a suspension of liposomes, and the size distribution, are commercially available. These typically use the techniques of dynamic light scattering and/or single-particle optical sensing e.g. the Accusizer™ and Nicomp™ series of instruments available from Particle Sizing Systems (Santa Barbara, USA), or the Zetasizer™ instruments from Malvern Instruments (UK), or the Particle Size Distribution Analyzer instruments from Horiba (Kyoto, Japan). Dynamic light scattering is the preferred method by which liposome diameters are determined. For a population of liposomes, the preferred method for defining the average liposome diameter in a composition of the invention is a Z-average i.e. the intensity-weighted mean hydrodynamic size of the ensemble collection of liposomes measured by dynamic light scattering (DLS). The Z-average is derived from cumulants analysis of the measured correlation curve, wherein a single particle size (liposome diameter) is assumed and a single exponential fit is applied to the autocorrelation function. The cumulants analysis algorithm does not yield a distribution but, in addition to an intensity-weighted Z-average, gives a polydispersity index.

Techniques for preparing suitable liposomes are well known in the art e.g. see Liposomes: Methods and Protocols, Volume 1: Pharmaceutical Nanocarriers: Methods and Protocols. (ed. Weissig). Humana Press, 2009. ISBN 160327359X, Liposome Technology, volumes I, II & III. (ed. Gregoriadis). Informa Healthcare, 2006, and Functional Polymer Colloids and Microparticles volume 4 (Microspheres, microcapsules & liposomes). (eds. Arshady & Guyot). Citus Books, 2002. One useful method is described in Jeffs et al. (2005) Pharmaceutical Research 22 (3):362-372, and involves mixing (i) an ethanolic solution of the lipids (ii) an aqueous solution of the nucleic acid and (iii) buffer, followed by mixing, equilibration, dilution and purification. Preferred liposomes for use with the invention are obtainable by this mixing process.

In certain embodiments, RNA is encapsulated within the liposomes, and so the liposome forms a outer layer around an aqueous RNA-containing core. This encapsulation has been found to protect RNA from RNase digestion. The liposomes can include some external RNA (e.g. on the surface of the liposomes), but at least half of the RNA (and ideally all of it) is encapsulated.

Useful compositions can include liposomes and RNA with a N:P ratio of between 1:1 and 20:1, where the “N:P ratio” is the molar ratio of nitrogen atoms in the cationic lipid to phosphates in the RNA (see WO2013/006825) e.g. a N:P ratio of 2:1, 4:1, 8:1 or 10:1.

### Polymeric Microparticles

Various polymers can form microparticles to encapsulate or adsorb RNA e.g. see WO2012/006359. The use of a substantially non-toxic polymer means that a recipient can safely receive the particles, and the use of a biodegradable polymer means that the particles can be metabolised after delivery to avoid long-term persistence. Useful polymers are also sterilisable, to assist in preparing pharmaceutical grade formulations.

Suitable non-toxic and biodegradable polymers include, but are not limited to, poly(α-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

In some embodiments, the microparticles are formed from poly(α-hydroxy acids), such as a poly(lactides) (“PLA”), copolymers of lactide and glycolide such as a poly(D,L-lactide-co-glycolide) (“PLG”), and copolymers of D,L-lactide and caprolactone. Useful PLG polymers include those having a lactide/glycolide molar ratio ranging, for example, from 20:80 to 80:20 e.g. 25:75, 40:60, 45:55, 50:50, 55:45, 60:40, 75:25. Useful PLG polymers include those having a molecular weight between, for example, 5,000-200,000 Da e.g. between 10,000-100,000, 20,000-70,000, 30,000-40,000, 40,000-50,000 Da.

The microparticles ideally have a diameter in the range of 0.02 μm to 8 μm. For a composition comprising a population of microparticles with different diameters at least 80% by number should have diameters in the range of 0.03-7 μm.

Techniques for preparing suitable microparticles are well known in the art e.g. see Arshady & Guyot, Polymers in Drug Delivery. (eds. Uchegbu & Schatzlein, CRC Press, 2006) in particular chapter 7, and Microparticulate Systems for the Delivery of Proteins and Vaccines. (eds. Cohen & Bernstein). CRC Press, 1996. To facilitate adsorption of RNA, a microparticle may include a cationic surfactant and/or lipid e.g. as disclosed in O'Hagan et al. (2001) J Virology 75:9037-9043 and Singh et al. (2003) Pharmaceutical Research 20: 247-251. An alternative way of making polymeric microparticles is by molding and curing e.g. as disclosed in WO2009/132206.

Microparticles of the invention can have a zeta potential of between 40-100 mV.

One advantage of microparticles over liposomes is that they are readily lyophilised for stable storage.

RNA can be adsorbed to the microparticles, and adsorption is facilitated by including cationic materials (e.g. cationic lipids) in the microparticle.

### Oil-in-Water Cationic Emulsions

Oil-in-water emulsions are known for adjuvanting protein-based influenza vaccines e.g. the MF59™ adjuvant in the FLUAD™ product, and the AS03 adjuvant in the PREPANDRIX™ product. RNA delivery according to the present invention can utilise an oil-in-water emulsion, provided that the emulsion includes one or more cationic molecules (see WO2012/006380, WO2013/006834 and WO2013/006837). For instance, a cationic lipid can be included in the emulsion to provide a positive droplet surface to which negatively-charged RNA can attach.

The emulsion comprises one or more oils. Suitable oil(s) include those from, for example, an animal (such as fish) or a vegetable source. The oil is ideally biodegradable (metabolisable) and biocompatible. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolisable and so may be used. The procedures for separation, purification, saponification and other means necessary for obtaining pure oils from animal sources are well known in the art.

Most fish contain metabolisable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Preferred emulsions comprise squalene, a shark liver oil which is a branched, unsaturated terpenoid. Squalane, the saturated analog to squalene, can also be used. Fish oils, including squalene and squalane, are readily available from commercial sources or may be obtained by methods known in the art.

Other useful oils are the tocopherols, particularly in combination with squalene. Where the oil phase of an emulsion includes a tocopherol, any of the α, β, γ, δ, ε or ξ tocopherols can be used, but α-tocopherols are preferred. D-α-tocopherol and DL-α-tocopherol can both be used. A preferred α-tocopherol is DL-α-tocopherol. An oil combination comprising squalene and a tocopherol (e.g. DL-α-tocopherol) can be used.

The oil in the emulsion may comprise a combination of oils e.g. squalene and at least one other oil.

The aqueous component of the emulsion can be plain water (e.g. w.f.i.) or can include further components e.g. solutes. For instance, it may include salts to form a buffer e.g. citrate or phosphate salts, such as sodium salts. Typical buffers include: a phosphate buffer; a Tris buffer; a borate buffer; a succinate buffer; a histidine buffer; or a citrate buffer. A buffered aqueous phase is preferred, and buffers will typically be included in the 5-20 mM range.

The emulsion also includes a cationic lipid. In certain embodiments this lipid is a surfactant so that it can facilitate formation and stabilisation of the emulsion. Useful cationic lipids generally contains a nitrogen atom that is positively charged under physiological conditions e.g. as a tertiary or quaternary amine. This nitrogen can be in the hydrophilic head group of an amphiphilic surfactant. Useful cationic lipids include, but are not limited to: 1,2-dioleoyloxy-3-(trimethylammonio)propane (DOTAP), 3′-[N—(N′,N′-Dimethylaminoethane)-carbamoyl] Cholesterol (DC Cholesterol), dimethyldioctadecyl-ammonium (DDA e.g. the bromide), 1,2-Dimyristoyl-3-Trimethyl-AmmoniumPropane (DMTAP), dipalmitoyl(C16:0)trimethyl ammonium propane (DPTAP), distearoyltrimethylammonium propane (DSTAP). Other useful cationic lipids are: benzalkonium chloride (BAK), benzethonium chloride, cetramide (which contains tetradecyltrimethylammonium bromide and possibly small amounts of dedecyltrimethylammonium bromide and hexadecyltrimethyl ammonium bromide), cetylpyridinium chloride (CPC), cetyl trimethylammonium chloride (CTAC), N,N′,N′-polyoxyethylene (10)-N-tallow-1,3-diaminopropane, dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemetha-naminium chloride (DEBDA), dialkyldimethylammonium salts, [1-(2,3-dioleyloxy)-propyl]-N,N,N,trimethylammonium chloride, 1,2-diacyl-3-(trimethylammonio) propane (acyl group=dimyristoyl, dipalmitoyl, distearoyl, dioleoyl), 1,2-diacyl-3 (dimethylammonio)propane (acyl group=dimyristoyl, dipalmitoyl, distearoyl, dioleoyl), 1,2-dioleoyl-3-(4′-trimethyl-ammonio)butanoyl-sn-glycerol, 1,2-dioleoyl 3-succinyl-sn-glycerol choline ester, cholesteryl (4′-trimethylammonio) butanoate, N-alkyl pyridinium salts (e.g. cetylpyridinium bromide and cetylpyridinium chloride), N-alkylpiperidinium salts, dicationic bolaform electrolytes (C₁₂Me₆; C₁₂Bu₆), dialkylglycetylphosphorylcholine, lysolecithin, L-α dioleoyl-phosphatidylethanolamine, cholesterol hemisuccinate choline ester, lipopolyamines, including but not limited to dioctadecylamidoglycylspermine (DOGS), dipalmitoyl phosphatidylethanol-amidospermine (DPPES), lipopoly-L (or D)-lysine (LPLL, LPDL), poly (L (or D)-lysine conjugated to N-glutarylphosphatidylethanolamine, didodecyl glutamate ester with pendant amino group (C₁₂GluPhCₙN⁺), ditetradecyl glutamate ester with pendant amino group (C₁₂GluPhCₙN⁺), cationic derivatives of cholesterol, including but not limited to cholesteryl-3β-oxysuccinamidoethylenetrimethylammonium salt, cholesteryl-3β-oxysuccinamidoethylene-dimethylamine, cholesteryl-3β-carboxyamidoethylenetrimethylammonium salt, and cholesteryl-3β-carboxyamidoethylenedimethylamine. Other useful cationic lipids are described in US-2008/0085870 and US-2008/0057080.

In particular embodiments, the cationic lipid is biodegradable (metabolisable) and biocompatible.

In addition to the oil and cationic lipid, an emulsion can include a non-ionic surfactant and/or a zwitterionic surfactant. Such surfactants include, but are not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy(oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); polyoxyethylene-9-lauryl ether; and sorbitan esters (commonly known as the Spans), such as sorbitan trioleate (Span 85) and sorbitan monolaurate. Preferred surfactants for including in the emulsion are polysorbate 80 (Tween 80; polyoxyethylene sorbitan monooleate), Span 85 (sorbitan trioleate), lecithin and Triton X-100.

Mixtures of these surfactants can be included in the emulsion e.g. Tween 80/Span 85 mixtures, or Tween 80/Triton-X100 mixtures. A combination of a polyoxyethylene sorbitan ester such as polyoxyethylene sorbitan monooleate (Tween 80) and an octoxynol such as t-octylphenoxy-polyethoxyethanol (Triton X-100) is also suitable. Another useful combination comprises laureth 9 plus a polyoxyethylene sorbitan ester and/or an octoxynol. Useful mixtures can comprise a surfactant with a HLB value in the range of 10-20 (e.g. polysorbate 80, with a HLB of 15.0) and a surfactant with a HLB value in the range of 1-10 (e.g. sorbitan trioleate, with a HLB of 1.8).

Preferred amounts of oil (% by volume) in the final emulsion are between 2-20% e.g. 5-15%, 6-14%, 7-13%, 8-12%. A squalene content of about 4-6% or about 9-11% is particularly useful.

Preferred amounts of surfactants (% by weight) in the final emulsion are between 0.001% and 8%. For example: polyoxyethylene sorbitan esters (such as polysorbate 80) 0.2 to 4%, in particular between 0.4-0.6%, between 0.45-0.55%, about 0.5% or between 1.5-2%, between 1.8-2.2%, between 1.9-2.1%, about 2%, or 0.85-0.95%, or about 1%; sorbitan esters (such as sorbitan trioleate) 0.02 to 2%, in particular about 0.5% or about 1%; octyl- or nonylphenoxy polyoxyethanols (such as Triton X-100) 0.001 to 0.1%, in particular 0.005 to 0.02%; polyoxyethylene ethers (such as laureth 9) 0.1 to 8%, particularly 0.1 to 10% and in particular 0.1 to 1% or about 0.5%.

The absolute amounts of oil and surfactant, and their ratio, can be varied within wide limits while still forming an emulsion. A skilled person can easily vary the relative proportions of the components to obtain a desired emulsion, but a weight ratio of between 4:1 and 5:1 for oil and surfactant is typical (excess oil).

An important parameter for ensuring immunostimulatory activity of an emulsion, particularly in large animals, is the oil droplet size (diameter). The most effective emulsions have a droplet size in the submicron range. Suitably the droplet sizes will be in the range 50-750 nm. Most usefully the average droplet size is less than 250 nm e.g. less than 200 nm, less than 150 nm. The average droplet size is usefully in the range of 80-180 nm. Ideally, at least 80% (by number) of the emulsion's oil droplets are less than 250 nm in diameter, and particularly at least 90%. Apparatuses for determining the average droplet size in an emulsion, and the size distribution, are commercially available. These typically use the techniques of dynamic light scattering and/or single-particle optical sensing e.g. the Accusizer™ and Nicomp™ series of instruments available from Particle Sizing Systems (Santa Barbara, USA), or the Zetasizer™ instruments from Malvern Instruments (UK), or the Particle Size Distribution Analyzer instruments from Horiba (Kyoto, Japan).

Ideally, the distribution of droplet sizes (by number) has only one maximum i.e. there is a single population of droplets distributed around an average (mode), rather than having two maxima. Preferred emulsions have a polydispersity of <0.4 e.g. 0.3, 0.2, or less.

Suitable emulsions with submicron droplets and a narrow size distribution can be obtained by the use of microfluidisation. This technique reduces average oil droplet size by propelling streams of input components through geometrically fixed channels at high pressure and high velocity. These streams contact channel walls, chamber walls and each other. The results shear, impact and cavitation forces cause a reduction in droplet size. Repeated steps of microfluidisation can be performed until an emulsion with a desired droplet size average and distribution are achieved.

As an alternative to microfluidisation, thermal methods can be used to cause phase inversion, as disclosed in US2007/0014805. These methods can also provide a submicron emulsion with a tight particle size distribution.

Preferred emulsions can be filter sterilised i.e. their droplets can pass through a 220 nm filter. As well as providing a sterilisation, this procedure also removes any large droplets in the emulsion.

In certain embodiments, the cationic lipid in the emulsion is DOTAP. The cationic oil-in-water emulsion may comprise from about 0.5 mg/ml to about 25 mg/ml DOTAP. For example, the cationic oil-in-water emulsion may comprise DOTAP at from about 0.5 mg/ml to about 25 mg/ml. In an exemplary embodiment, the cationic oil-in-water emulsion comprises from about 0.8 mg/ml to about 1.6 mg/ml DOTAP, such as 0.8 mg/ml, 1.2 mg/ml, 1.4 mg/ml or 1.6 mg/ml.

In certain embodiments, the cationic lipid is DC Cholesterol. The cationic oil-in-water emulsion may comprise DC Cholesterol at from about 0.1 mg/ml to about 5 mg/ml DC Cholesterol. For example, the cationic oil-in-water emulsion may comprise DC Cholesterol from about 0.1 mg/ml to about 5 mg/ml. In an exemplary embodiment, the cationic oil-in-water emulsion comprises from about 0.62 mg/ml to about 4.92 mg/ml DC Cholesterol, such as 2.46 mg/ml.

In certain embodiments, the cationic lipid is DDA. The cationic oil-in-water emulsion may comprise from about 0.1 mg/ml to about 5 mg/ml DDA. For example, the cationic oil-in-water emulsion may comprise DDA at from about 0.1 mg/ml to about 25 mg/ml. In an exemplary embodiment, the cationic oil-in-water emulsion comprises from about 0.73 mg/ml to about 1.45 mg/ml DDA, such as 1.45 mg/ml.

Certain preferred compositions of the invention for administration to a patient comprise squalene, span 85, polysorbate 80, and DOTAP. For instance: squalene may be present at 5-15 mg/ml; span 85 may be present at 0.5-2 mg/ml; polysorbate 80 may be present at 0.5-2 mg/ml; and DOTAP may be present at 0.1-10 mg/ml. The emulsion can include the same amount (by volume) of span 85 and polysorbate 80. The emulsion can include more squalene than surfactant. The emulsion can include more squalene than DOTAP.

### The Immunogenic Composition

Immunogenic compositions will typically include a pharmaceutically acceptable carrier in addition to RNA and polypeptide (and any delivery system). A thorough discussion of such carriers is available in Gennaro (2000) Remington: The Science and Practice of Pharmacy, 20th edition.

Pharmaceutical compositions of the invention may include the active components (RNA and polypeptide) in plain water (e.g. w.f.i.) or in a buffer e.g. a phosphate buffer, a Tris buffer, a borate buffer, a succinate buffer, a histidine buffer, or a citrate buffer. Buffer salts will typically be included in the 5-20 mM range.

Pharmaceutical compositions of the invention may have a pH between 5.0 and 9.5 e.g. between 6.0 and 8.0.

Compositions of the invention may include sodium salts (e.g. sodium chloride) to give tonicity. A concentration of 10±2 mg/ml NaCl is typical e.g. about 9 mg/ml.

Compositions of the invention may include metal ion chelators. These can prolong RNA stability by removing ions which can accelerate phosphodiester hydrolysis. Thus a composition may include one or more of EDTA, EGTA, BAPTA, pentetic acid, etc. Such chelators are typically present at between 10-500 μM e.g. 0.1 mM. A citrate salt, such as sodium citrate, can also act as a chelator, while advantageously also providing buffering activity.

Pharmaceutical compositions of the invention may have an osmolality of between 200 mOsm/kg and 400 mOsm/kg, e.g. between 240-360 mOsm/kg, or between 290-310 mOsm/kg.

Pharmaceutical compositions of the invention may include one or more preservatives, such as thiomersal or 2-phenoxyethanol. Mercury-free compositions are preferred, and preservative-free vaccines can be prepared.

Pharmaceutical compositions of the invention are in particular sterile.

Pharmaceutical compositions of the invention are in particular non-pyrogenic e.g. containing <1 EU (endotoxin unit, a standard measure) per dose, and particularly <0.1 EU per dose.

In certain embodiments, pharmaceutical compositions of the invention are gluten free.

Pharmaceutical compositions of the invention may be prepared in unit dose form. In some embodiments a unit dose may have a volume of between 0.1-1.0 ml e.g. about 0.5 ml.

A pharmaceutical composition of the invention may include one or more small molecule immunopotentiators. For example, the composition may include a TLR2 agonist (e.g. Pam3CSK4), a TLR4 agonist (e.g. an aminoalkyl glucosaminide phosphate, such as E6020), a TLR7 agonist (e.g. imiquimod), a TLR8 agonist (e.g. resiquimod) and/or a TLR9 agonist (e.g. IC31). Any such agonist ideally has a molecular weight of <2000 Da.

The compositions may be prepared as injectables, either as solutions or suspensions. The composition may be prepared for pulmonary administration e.g. by an inhaler, using a fine spray. The composition may be prepared for nasal, aural or ocular administration e.g. as spray or drops. Injectables for intramuscular administration are typical.

Compositions comprise an immunologically effective amount of RNA and polypeptide, as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. The polypeptide and RNA content of compositions of the invention will generally be expressed in terms of the amount of RNA per dose. A preferred dose has ≦100 μg RNA (e.g. from 10-100 μg, such as about 10 μg, 25 μg, 50 μg, 75 μg or 100 μg). Expression can be seen at much lower levels (e.g. ≦1 μg/dose, ≦100 ng/dose, ≦10 ng/dose, ≦1 ng/dose), but a minimum dose of 0.1 μg is preferred (see WO2012/006369).

The invention also provides a delivery device (e.g. syringe, nebuliser, sprayer, inhaler, dermal patch, etc.) containing a pharmaceutical composition of the invention. This device can be used to administer the composition to a subject.

### Methods of Treatment and Medical Uses

Pharmaceutical compositions of the invention are for in vivo use for eliciting an immune response against influenza virus.

The invention provides a method for raising an immune response in a vertebrate comprising the step of administering an effective amount of a pharmaceutical composition of the invention. In certain embodiments, the immune response is protective and in certain aspects, involves antibodies and/or cell-mediated immunity. The method may raise a booster response.

The invention also provides a pharmaceutical composition of the invention for use in a method for raising an immune response against influenza virus in a vertebrate.

The invention also provides the use of a RNA molecule and polypeptide, as described above, in the manufacture of a medicament for raising an immune response against influenza virus in a vertebrate.

By raising an immune response in the vertebrate by these uses and methods, the vertebrate can be protected against influenza virus infection and/or disease. The compositions are immunogenic, and in certain embodiments are vaccine compositions. Vaccines according to the invention may either be prophylactic (i.e. to prevent infection) or therapeutic (i.e. to treat infection), but will typically be prophylactic.

The vertebrate is in particular embodiments a mammal, such as a human or a large veterinary mammal (e.g. horses, cattle, deer, goats, pigs). Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc.

Vaccines prepared according to the invention may be used to treat both children and adults. Thus a human patient may be less than 1 year old, less than 5 years old, 1-5 years old, 5-15 years old, 15-55 years old, or at least 55 years old. Preferred patients for receiving the vaccines are the elderly (e.g. ≧50 years old, ≧60 years old, and particularly ≧65 years), the young (e.g. ≦5 years old), hospitalised patients, healthcare workers, armed service and military personnel, pregnant women, the chronically ill, or immunodeficient patients. The vaccines are not suitable solely for these groups, however, and may be used more generally in a population.

Compositions of the invention will generally be administered directly to a patient. Direct delivery may be accomplished by parenteral injection (e.g. subcutaneously, intraperitoneally, intravenously, intramuscularly, intradermally, or to the interstitial space of a tissue). Alternative delivery routes include rectal, oral (e.g. tablet, spray), buccal, sublingual, vaginal, topical, transdermal or transcutaneous, intranasal, ocular, aural, pulmonary or other mucosal administration. Intradermal and intramuscular administration are two preferred routes. Injection may be via a needle (e.g. a hypodermic needle), but needle-free injection may alternatively be used. A typical intramuscular dose is 0.5 ml.

The invention may be used to elicit systemic and/or mucosal immunity, and in particular to elicit an enhanced systemic and/or mucosal immunity.

One way of checking efficacy of therapeutic treatment involves monitoring pathogen infection after administration of the composition. One way of checking efficacy of prophylactic treatment involves monitoring immune responses, systemically (such as monitoring the level of IgG1 and IgG2a production) and/or mucosally (such as monitoring the level of IgA production), against the antigen. Typically, antigen-specific serum antibody responses are determined post-immunization. Another way of assessing the immunogenicity of the compositions is to screen patient sera or mucosal secretions against a target polypeptide. A positive reaction between the protein and the patient sample indicates that the patient has mounted an immune response to the polypeptide in question. The efficacy of the compositions can also be determined in vivo by challenging appropriate animal models of the pathogen of interest infection.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.). In one embodiment, multiple doses may be administered approximately 6 weeks, 10 weeks and 14 weeks after birth, e.g. at an age of 6 weeks, 10 weeks and 14 weeks, as often used in the World Health Organisation's Expanded Program on Immunisation (“EPI”). In an alternative embodiment, two primary doses are administered about two months apart, e.g. about 7, 8 or 9 weeks apart, followed by one or more booster doses about 6 months to 1 year after the second primary dose, e.g. about 6, 8, 10 or 12 months after the second primary dose. In a further embodiment, three primary doses are administered about two months apart, e.g. about 7, 8 or 9 weeks apart, followed by one or more booster doses about 6 months to 1 year after the third primary dose, e.g. about 6, 8, 10, or 12 months after the third primary dose.

### Kits

The invention also provides a kit comprising (a) a first kit component comprising a polypeptide that comprises an epitope from an influenza virus antigen, and (b) a second kit component comprising a self-replicating RNA which encodes a polypeptide that comprises an epitope from an influenza virus antigen.

In one aspect, the two kit components can be mixed to give an immunogenic composition of the invention. In another aspect, the kit is suitable for administering an immunisation regimen in which the first component is administered before the second composition, to generate an immune response against influenza virus.

The first and second kit components are stored separately. Their containers can be separate from each other (e.g. two vials) or joined to each other (e.g. two chambers in a dual-chamber syringe).

Either or both of the kit components can be in aqueous form. Either or both of the kit components can be in solid or dry form (e.g. lyophilized).

When the RNA and polypeptide are co-administered, it may still be desirable to package and store them separately. The two components may be combined, e.g., within about 72 hours, about 48 hours, about 24 hours, about 12 hours, about 10 hours, about 9 hours, about 8 hours, about 7 hours, about 6 hours, about 5 hours, about 4 hours, about 3 hours, about 2 hours, about 1 hour, about 45 minutes, about 30 minutes, about 15 minutes, about 10 minutes, or about 5 minutes prior to administration. For example, the polypeptide and RNA can be combined at a patient's bedside.

Where the components are administered in sequence, they may be administered within about 4 hours, about 3 hours, about 2 hours, about 1 hour, about 45 minutes, about 30 minutes, about 15 minutes, about 10 minutes, or about 5 minutes of each other. The priming composition, the boosting composition, or both, may optionally include one or more delivery systems, immunoregulatory agents such as adjuvants, etc. as described herein.

Suitable containers for kit components include, for example, bottles, vials, syringes, and test tubes. Containers can be formed from a variety of materials, including glass or plastic. A container may have a sterile access port (for example, the container may be an intravenous solution bag or a vial having a stopper piercable by a hypodermic injection needle).

The kit can further comprise a third container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may further include a fourth container comprising an adjuvant (such as an oil-in-water emulsion).

The kit can also comprise a package insert containing written instructions for methods of inducing immunity or for treating infections. The package insert can be an unapproved draft package insert or can be a package insert approved by the Food and Drug Administration (FDA) or other regulatory body.

One aspect of the invention relates to the “prime and boost” immunization regimens in which the immune response induced by a priming composition is boosted by a boosting composition. For example, following priming (at least once) with an antigen (e.g., after administration of RNA or polypeptide), a boosting composition comprising substantially a different form of the antigen (e.g. RNA instead of polypeptide, or vice versa). Administration of the boosting composition is generally weeks or months after administration of the priming composition, such as about 1 week, about 2 weeks, about 3 weeks, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1 month, about 2 months, about 3 months, about 4 months, about 5 months, about 6 months, about 7 months, about 8 months, about 9 months, about 10 months, about 11 months, about 12 months, about 18 months, about 2 years, about 3 years, about 4 years, about 5 years, about 6 years, about 7 years, about 8 years, about 9 years, or about 10 years after the priming composition is administered.

### General

The term “comprising” encompasses “including” as well as “consisting” e.g. a composition “comprising” X may consist exclusively of X or may include something additional e.g. X+Y Y.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

An “epitope” is a portion of an antigen that is recognized by the immune system (e.g., by an antibody, or by a T cell receptor). A polypeptide epitope can be linear or conformational. T-cells and B-cells recognize antigens in different ways. T-cells recognize peptide fragments of proteins that are embedded in class-II or class-I MHC molecules at the surface of cells, whereas B-cells recognize surface features of an unprocessed antigen, via immunoglobulin-like cell surface receptors. The difference in antigen recognition mechanisms of T-cells and B-cells are reflected in the different natures of their epitopes. Thus, whereas B-cells recognize surface features of an antigen or a pathogen, T-cell epitopes (which comprise peptides of about 8-12 amino acids in length) can be “internal” as well as “surface” when viewed in the context of the three-dimensional structure of the antigen. Accordingly, a B-cell epitope is preferably exposed on the surface of the antigen or pathogen, and can be linear or conformational, whereas a T-cell epitope is typically linear but is not required to be available or on the surface of the antigen. Normally, a B-cell epitope will include at least about 5 amino acids but can be as small as 3-4 amino acids. A T-cell epitope, such as a CTL epitope, will typically include at least about 7-9 amino acids, and a helper T-cell epitope will typically include at least about 12-20 amino acids.

When an individual is immunized with a polypeptide antigen having multiple epitopes, in many instances the majority of responding T lymphocytes will be specific for one or a few linear epitopes from that antigen and/or a majority of the responding B lymphocytes will be specific for one or a few linear or conformational epitopes from that antigen. Such epitopes are typically referred to as “immunodominant epitopes.” In an antigen having several immunodominant epitopes, a single epitope may be most dominant, and is typically referred to as the “primary” immunodominant epitope. The remaining immunodominant epitopes are typically referred to as “secondary” immunodominant epitope(s).

### MODES FOR CARRYING OUT THE INVENTION

Balb/C mice were immunised with hemagglutinin from Influenza A/Turkey/Turkey/2005 (H5N1). Compositions were administered at days 0 and 56, and serum was sampled at days 0, 21, 56 and 72. Hemagglutinin was delivered either as protein or encoded within a self-replicating alphavirus RNA replicon. RNA was delivered with a cationic nanoemulsion (CNE), and protein was delivered either in buffer or with an oil-in-water emulsion adjuvant (MF59). Controls received ovalbumin or buffer (PBS) alone. Mice were in ten groups, 12 mice per group:

| Group | Day 0 | Day 56 |
|---|---|---|
| 1 | PBS | PBS |
| 2 | OVA(RNA), 1 μg in CNE | |
| 3 | Replicon, 1 μg in CNE | Replicon, 1 μg in CNE |
| 4 | Replicon, 10 μg in CNE | Replicon, 10 μg in CNE |
| 5 | Replicon, 10 μg in CNE | Protein, 1 μg in PBS |
| 6 | Replicon, 10 μg in CNE | Protein, 0.1 μg in MF59 |
| 7 | Protein, 1 μg in PBS | Protein, 1 μg in PBS |
| 8 | Protein, 1 μg in MF59 | Protein, 1 μg in MF59 |
| 9 | Protein 0.1 μg + replicon | Protein 0.1 μg + replicon |
| | 10 μg in CNE | 10 μg in CNE |
| 10 | Protein, 0.1 μg in CNE | Protein, 0.1 μg in CNE |

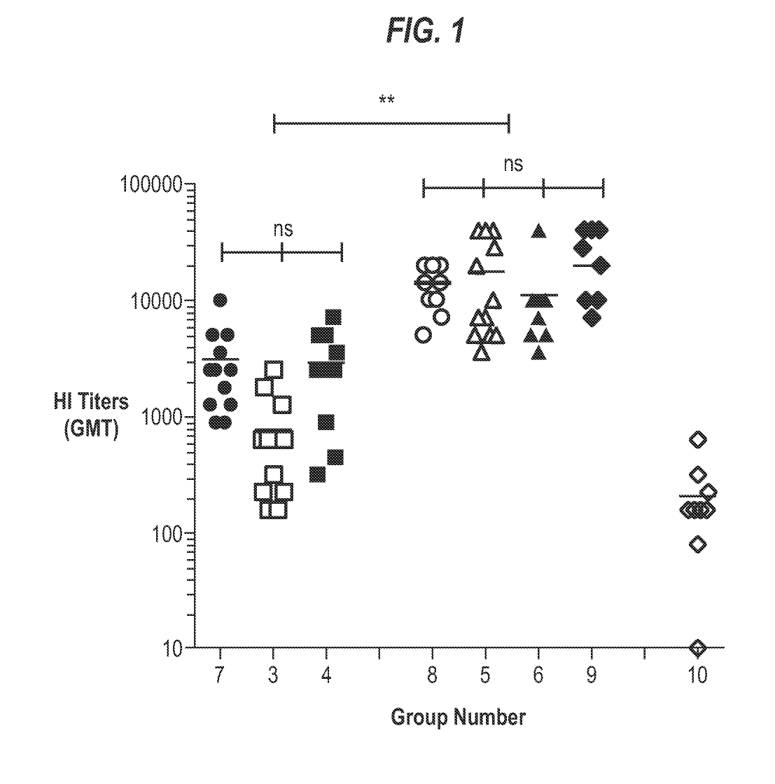

FIG. 1 shows hemagglutination inhibition (HI) titers at day 72. The mixture of RNA and protein (group 9) showed a titer as high as MF59-adjuvanted protein (group 8). A similar effect was seen in a microneutralisation test, where titers against three different H5N1 strains were again comparable to those obtained using MF59-adjuvanted protein.

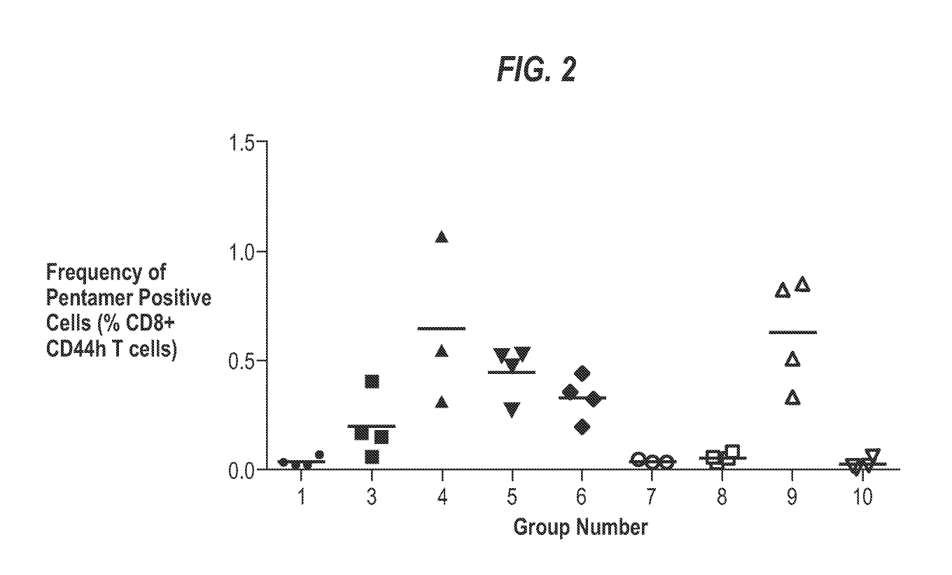

CD8+ T cells were measured on day 105 using MHCI pentamer specific for the HA₅₃₃₋₅₄₁ peptide. This peptide is conserved between H1 and H5 strains. The results in FIG. 2 show that the use of a mixed RNA/protein composition caused antigen-specific T cells to remain in circulation for a long time after immunisation.

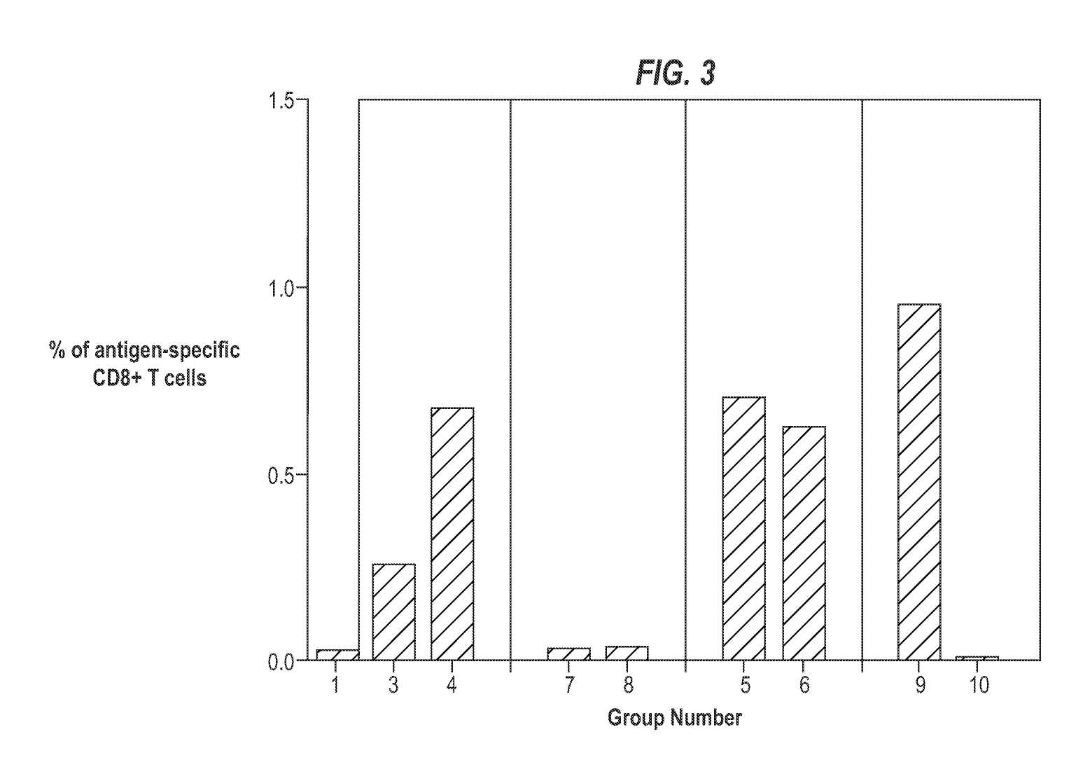

FIG. 3 shows H5-specific Cd8+ T cell responses 12 weeks after the second dose. Group 9 shows the highest proportion of antigen-specific CD8+ T cells.

It will be understood that the invention has been described by way of example only and modifications may be made whilst remaining within the scope and spirit of the invention.

### Table 1: Useful Phospholipids

DDPC 1,2-Didecanoyl-sn-Glycero-3-phosphatidylcholine

### DEPA 1,2-Dierucoyl-sn-Glycero-3-Phosphate

DEPC 1,2-Erucoyl-sn-Glycero-3-phosphatidylcholine
DEPE 1,2-Dierucoyl-sn-Glycero-3-phosphatidylethanolamine
DEPG 1,2-Dierucoyl-sn-Glycero-3 [Phosphatidyl-rac-(1-glycerol . . . )
DLOPC 1,2-Linoleoyl-sn-Glycero-3-phosphatidylcholine

### DLPA 1,2-Dilauroyl-sn-Glycero-3-Phosphate

DLPC 1,2-Dilauroyl-sn-Glycero-3-phosphatidylcholine
DLPE 1,2-Dilauroyl-sn-Glycero-3-phosphatidylethanolamine
DLPG 1,2-Dilauroyl-sn-Glycero-3 [Phosphatidyl-rac-(1-glycerol . . . )
DLPS 1,2-Dilauroyl-sn-Glycero-3-phosphatidylserine
DMG 1,2-Dimyristoyl-sn-glycero-3-phosphoethanolamine

### DMPA 1,2-Dimyristoyl-sn-Glycero-3-Phosphate

DMPC 1,2-Dimyristoyl-sn-Glycero-3-phosphatidylcholine
DMPE 1,2-Dimyristoyl-sn-Glycero-3-phosphatidylethanolamine
DMPG 1,2-Myristoyl-sn-Glycero-3 [Phosphatidyl-rac-(1-glycerol . . . )
DMPS 1,2-Dimyristoyl-sn-Glycero-3-phosphatidylserine

### DOPA 1,2-Dioleoyl-sn-Glycero-3-Phosphate

DOPC 1,2-Dioleoyl-sn-Glycero-3-phosphatidylcholine
DOPE 1,2-Dioleoyl-sn-Glycero-3-phosphatidylethanolamine
DOPG 1,2-Dioleoyl-sn-Glycero-3 [Phosphatidyl-rac-(1-glycerol . . . )
DOPS 1,2-Dioleoyl-sn-Glycero-3-phosphatidylserine

### DPPA 1,2-Dipalmitoyl-sn-Glycero-3-Phosphate

DPPC 1,2-Dipalmitoyl-sn-Glycero-3-phosphatidylcholine
DPPE 1,2-Dipalmitoyl-sn-Glycero-3-phosphatidylethanolamine
DPPG 1,2-Dipalmitoyl-sn-Glycero-3 [Phosphatidyl-rac-(1-glycerol . . . )
DPPS 1,2-Dipalmitoyl-sn-Glycero-3-phosphatidylserine
DPyPE 1,2-diphytanoyl-sn-glycero-3-phosphoethanolamine

### DSPA 1,2-Distearoyl-sn-Glycero-3-Phosphate

DSPC 1,2-Distearoyl-sn-Glycero-3-phosphatidylcholine
DSPE 1,2-Diostearpyl-sn-Glycero-3-phosphatidylethanolamine
DSPG 1,2-Distearoyl-sn-Glycero-3 [Phosphatidyl-rac-(1-glycerol . . . )
DSPS 1,2-Distearoyl-sn-Glycero-3-phosphatidylserine

### EPC Egg-PC

### HEPC Hydrogenated Egg PC

HSPC High purity Hydrogenated Soy PC

### HSPC Hydrogenated Soy PC

LYSOPC MYRISTIC 1-Myristoyl-sn-Glycero-3-phosphatidylcholine
LYSOPC PALMITIC 1-Palmitoyl-sn-Glycero-3-phosphatidylcholine
LYSOPC STEARIC 1-Stearoyl-sn-Glycero-3-phosphatidylcholine
Milk Sphingomyelin MPPC 1-Myristoyl,2-palmitoyl-sn-Glycero 3-phosphatidylcholine
MSPC 1-Myristoyl,2-stearoyl-sn-Glycero-3-phosphatidylcholine
PMPC 1-Palmitoyl,2-myristoyl-sn-Glycero-3-phosphatidylcholine
POPC 1-Palmitoyl,2-oleoyl-sn-Glycero-3-phosphatidylcholine
POPE 1-Palmitoyl-2-oleoyl-sn-Glycero-3-phosphatidylethanolamine
POPG 1,2-Dioleoyl-sn-Glycero-3 [Phosphatidyl-rac-(1-glycerol).]
PSPC 1-Palmitoyl,2-stearoyl-sn-Glycero-3-phosphatidylcholine
SMPC 1-Stearoyl,2-myristoyl-sn-Glycero-3-phosphatidylcholine
SOPC 1-Stearoyl,2-oleoyl-sn-Glycero-3-phosphatidylcholine
SPPC 1-Stearoyl,2-palmitoyl-sn-Glycero-3-phosphatidylcholine

1. An immunogenic composition comprising: (i) a self-replicating RNA molecule that encodes a first polypeptide antigen comprising a first epitope from an influenza virus antigen; and (ii) a second polypeptide antigen comprising a second epitope from an influenza virus antigen; wherein:
(a) the first and second epitopes are both from influenza hemagglutinin;
(b) the first and second epitopes are both from influenza A virus; and/or
(c) the first epitope and the second epitope are both from influenza B virus.

2. The immunogenic composition of claim 1, wherein said first and second epitopes are both from an influenza A virus hemagglutinin.

3. The immunogenic composition of claim 2, wherein said first and second epitopes are both from the same subtype of an influenza A virus hemagglutinin.

4. The immunogenic composition of claim 2, wherein said first and second epitopes are both from influenza A virus H5 hemagglutinin.

5. The immunogenic composition of claim 3, wherein said first and second epitopes are both from the same influenza A virus hemagglutinin.

6. The immunogenic composition of claim 1, wherein said first and second epitopes are both from an influenza B virus hemagglutinin.

7. The immunogenic composition of claim 6, wherein said first and second epitopes are both from the same lineage of an influenza B virus hemagglutinin.

8. The immunogenic composition of claim 7, wherein said first and second epitopes are both from the same influenza B virus hemagglutinin.

9. The immunogenic composition of claim 1, wherein said first and second epitopes are the same.

10. The immunogenic composition of claim 1, wherein said first and second polypeptide antigens share at least two B-cell epitopes.

11. The immunogenic composition of claim 1, wherein (a) said first and second polypeptide antigens share a common amino acid sequence which includes multiple epitopes and is 80 amino acids or longer e.g. 120 amino acids or longer, and/or (b) said first and second polypeptide antigens have at least 80% amino acid sequence identity to each other.

12. The immunogenic composition of claim 1, wherein the self-replicating RNA is an alphavirus-derived RNA replicon.

13. The immunogenic composition of claim 1, wherein the self-replicating RNA molecule comprises one or more modified nucleotides.

14. The immunogenic composition of claim 1, including (i) liposomes (ii) non-toxic and biodegradable polymer microparticles or (iii) a cationic submicron oil-in-water emulsion.

15. A method for treating or preventing influenza disease and/or infection, comprising administering to a subject in need thereof a therapeutically effective amount of a composition of claim 1.

16. A method for inducing an immune response in a subject comprising administering to a subject in need thereof a therapeutically effective amount of the composition of claim 1.

17. A kit comprising (a) a first kit component comprising a polypeptide that comprises an epitope from an influenza virus antigen, and (b) a second kit component comprising a self-replicating RNA which encodes a polypeptide that comprises an epitope from an influenza virus antigen.
